(19) United States
(12) Patent Application Publication
(10) Pub. No.: US 2010/0083390 A1
(43) Pub. Date: Apr. 1, 2010

(54) FORMULATION FOR THE BIOLOGICAL CONTROL OF INSECT-PESTS

(75) Inventor: Abdulhamid H.K. Al-Amidi, Wicklow (IE)

Correspondence Address: KNOBBE MARTENS OLSON & BEAR LLP, 2040 MAIN STREET, FOURTEENTH FLOOR, IRVINE, CA, 92614, US

(73) Assignee: NEMOS HORTICULTURAL LIMITED, Dublin (IE)

(21) Appl. No.: 12/532,772

(22) PCT Filed: Mar. 25, 2008
(86) PCT No.: PCT/IE08/00027

§ 371 Date: Sep. 23, 2009

(30) Foreign Application Priority Data
Mar. 23, 2007 (EP) 07394004.1

(51) Int. Cl. A01K 67/00

(52) U.S. Cl. 800/8

(57) ABSTRACT

A formulation for the biological control of pests comprises a cohort of infective juvenile insect-parasitic nematodes from at least three species, wherein at least two of the species are of a first genus, and at least one of the species is from a second genus, and the species number from the first genus is greater than the species number from the second genus. The cohort of nematodes consists essentially of two species from the first genus and one species from the second genus and comprises at least one ambusher foraging species of nematode from a first genera and at least two cruiser foraging species of nematode from a different genera, or at least two ambusher foraging species of nematode from the same genera and at least one cruiser foraging species of nematode from a different genera. The first and second genus are selected from the group comprising: Steinernematidae and Heterorhapditidae.

The invention relates to a formulation for use in the biological control of insect pests comprising a cohort of infective juvenile insect-parasitic nematodes.

### BACKGROUND TO THE INVENTION

Entomopathogenic nematodes refer to nematodes that are parasitic to one or more species of insect. The most important Order of entomopathogenic nematodes is the Rhabditida, which contains several families, many of whose members are parasitic to insects. The Steinernematidae and the Heterorhabditidae are well known members of this order (Rhabditida). Both the families, Steinernematidae and Heterorhabditidae contain several species, each specialised for attacking a specific group of insect pests (Table 1). Although the two families are not closely related, phylogenetically, both share similar life histories. The cycle begins with an infective juvenile (non-feeding juvenile), whose only function is to seek out and infect new hosts. After entering the insect via natural body openings, mouth, anus, spiracles or areas of thin cuticle, the infective juveniles release an associated mutualistic bacterium. These bacteria of the genus Xenorhabdus or heterorhabditids, respectively, cause host mortality within 48 hours.

The foraging strategies of entomopathogenic nematodes vary between species, and determine their soil depth distribution and host preference. In order to find hosts, infective juveniles use strategies are that vary based on ambush or cruise foraging or a combination of both. In order to ambush prey, some Steinernema species raise their bodies off the soil surface so they are better poised to attach to passing insects. Other species adopt a cruising strategy, they roam through the soil searching for potential hosts. These foraging strategies influence which hosts the nematodes infect. For example, ambush predators such as Steinernema carpocapsae infect more insects on the surface, while cruising predators like Heterorhabditis bacteriophora infect insects that live deep in the soil.

One of the major problems which arise with the use of existing, commercially available, biological control products, is that the products contain only one species of the above beneficial nematodes and, as such, are able to control only a single or one group of target insect species, make it uneconomically viable comparing with the use of chemical pesticide which control a wide range of insect species, beneficial or pests alike, in one application. Commercial growers will not adopt biological control agents that do not provide efficacy comparable with standard chemical insecticides.

Neumann et al. (2006) studied the interactions and spatial separation among one heterorhabditid and two steinernematid nematode species with different foraging strategies within a sand column. They introduced the nematodes 7 days before the host was introduced to the system. They found that each individual species performed better on its own than in a three species combination. They also found this to be the case at all depths, with the exception of the cruiser forager, H. bacteriophora, which performed better in combination than alone, but only at a deeper level. This is especially a problem as many of the insect pests targeted by domestic and commercial growers reside in a soil depth range of 0-25 cm.

It is an object of the invention to overcome at least one of the above problems.

### STATEMENTS OF INVENTION

The invention relates to a multispecies nematode formulation for killing target insect pests. The formulation has at least three species of nematode from at least two families (Steinernematidae and Heterorhabditidae), and is formulated such that the species from one family outnumber the species from the other family. Thus, the formulation includes a majority species and a minority species. An example would be a formulation comprising Steinernema riobravis, Heterorhabditis bacteriophora, and Heterorhabditis megidis, where Steinernema riobravis is the minority species. The Applicant has surprisingly discovered that in such a formulation, the [symbol] to competition provided by the presence of the majority species. Thus, formulations may be targeted to specific target pests by including as the minority species one (or more) species of nematode that are specialised parasites to the specific target pest.

According to the invention, there is provided a formulation for use in the biological control of insect pests comprising a cohort of infective juvenile insect-parasitic nematodes from at least three species, wherein at least two of the species are of a first genus (majority species), and at least one of the species are from a second genus (minority species), and wherein the number of species from the majority species is greater than the number of species from the minority species.

A formulation according to the invention provides a number of advantages over known nematode formulations.

First, as the formulation comprises a majority species and a minority species which are in competition for insect pests, the species present from the minority species will compete more aggressively for hosts that those from the first genus. This surprisingly results in the minority species being more effective in infecting and killing insect pests. Further, it allows the formulator to tailor any given formulation for effective killing of a predetermined target insect pest (or group of target pests) by including a species specifically directed to that target insect pest as the minority species. As an example, a formulation directed to treatment of root riobravis, Heterorhabditis bacteriophora, and Heterorhabditis megidis, where Steinernema riobravis is chosen as the minority species as it is a specialised parasite of the root weevil. In this formulation, as Steinernema riobravis is the representative of the minority species, it will compete more aggressively for hosts that those from the first genus (majority), and as such will have an increased killing power compared to single nematode species when applied on its own (as an individual application outside of the formula).

Secondly, the formulations of the invention, by virtue of including at least three different species of nematode, will effectively have a broad spectrum killing power by virtue of the fact that nematode species specialised in infective killing of different varieties of nematode will be included in the formulation. Thus, taking the example above, the three species present will have an effective killing power against the following list of insect pests: root weevils, tawny mole crickets, southern mole crickets, armyworm, cutworm, webworm, wood borers, artichoke plume moth, and scarab, with a particularly effective killing power against those pests for which the minority species is a specialised killer. Thus, the formulation of the invention provide a killing efficacy which is comparable to convention chemical pesticides. Thirdly, as the nematode species chosen for the formulation are highly specific parasites, they will not attack and kill beneficial insects. In contrast, chemical pesticides are unable to distinguish between beneficial insects and unwanted insect pests.

Without being bound by theory, it is also believed that the use of a multispecies nematode formulation according to the invention, in which a degree of competition exists between the minority and majority species, has the effect of controlling the population of nematodes and preventing an uncontrolled increase in the nematode population.

In one embodiment of the invention, the species of nematode present in the formulation will include (a) at least one cruiser foraging species and at least one ambusher foraging species, or (b) a nematode that is capable of both cruiser and ambusher foraging strategies. This allows the combined species of the formulation to have a broad foraging strategy. Thus, typically the formulation will include two ambusher foraging species from the same genera and one cruiser foraging species from a different genera, or two cruiser foraging species from the same genera and one ambusher foraging species from a different genera. In the specification, the term “cruiser foraging “species” is taken to mean a species of nematode that adopts a foraging strategy of roaming through the soil looking for potential hosts. Examples of cruiser foraging species include Heterorhabditis bacteriophora, Steinernema glaseri, Steinernema kraussei, Heterorhabditis megadis. In this specification, the term “ambusher foraging species” shall be taken to mean a species of nematode that adopts a foraging strategy of raising their bodies off the surface of the soil so that they are better disposed to attach to insect pests passing along the surface of the soil. Examples of ambusher foraging species include Steinernema carpocapsae, and are capable of both cruiser and ambusher foraging strategies are Steinernema riobravis, and Steinernema feltiae. Thus, a formulation according to this embodiment will have an advantage of being able to attack insect pests that inhabit above the soil as well as those that inhabit below the soil, thereby enhancing the broad spectrum nature of the formulation of the invention.

Surprisingly, the Applicant has shown that when the formulations of the invention employ species that have the same foraging strategy (i.e. all cruiser or all ambusher), that the presence of the majority and minority species has the effect of altering the foraging strategy of the species such that they adapts a broad foraging strategy. This will allow the formulator a degree of flexibility when formulating a mixture of nematode species to achieve a broad foraging strategy.

Typically, the cohort of infective juvenile nematodes consists essentially of two or more species from the first genus and one species from the second genus. Thus, for example, the cohort may comprise 3, 4, 5, 6, 7, 9, or 10 species from one genus, and one species from the second genus. Alternatively, the cohort may comprise n+1 number of species of the first genus, and n number of species from the second genus. Typically, n is less than 9, 6, 4 or 3. Alternatively, the cohort may comprise X number of species from the first genus, and Y number of species from the second genus, where X and Y are whole numbers and X>Y. Typically, X is less than 10, 7, (minority species) will be in a minority in the formulation, and will therefore have to compete more strongly than the species of the first genus (majority species). This results in the minority species having a grater destructive action, as when compared to the same single nematode species if applied on its own (that is to say, in a comparatively equal quantity, as an individual application outside of the formula, for example a species of Y individually and separately applied).

In a preferred embodiment of the invention, the cohort of infective juvenile nematodes consists essentially of two species from the first genus and one species from the second genus.

Typically, the first and second genus are selected from the two families comprising: Steinernematidae; and Heterorhapditidae.

In one embodiment of the invention, the first genus (majority species) is Steinernema (Steinernematidae) and the second genus (minority species) is Heterorhabditis (Heterorhapditidae). Typically, the two or more species of Steinernematidae are selected from the group comprising: Steinernema feltiae; Stinernema scapterisci; Steinernema riobravis; Steinernema carpocapsae; and Steinernema krussei. Ideally, the two species of Steinernematidae are selected from the group comprising Steinernema feltiae, Steinernema krussei, and Steinernema carpocapsae. [symbol] selected from the group comprising: Heterorhabditis megidis Heterorhabditis downesi; and Heterorhabditis bacteriophora. Ideally, the at least one species of Heterorhapditidae consists of Heterorhabditis bacteriophora.

In another embodiment of the invention, the first genus (majority species) is Heterorhabditis (Heterorhapditidae) and the second genus (minority species) is Steinernema (Steinernematidae). In this case, typically the two species of Heterorhapditidae are selected from the group comprising: Heterorhabditis megidis; Heterorhabditis downesi and Heterorhabditis bacteriophora. Suitably, the at least one species of Steinernematidae is selected from the group comprising: Steinernema feltiae; Stinernema scapterisci; Steinernema riobravis; Steinernema carpocapsae; and Steinernema krussei. Ideally, the at least one species of Steinernematidae is selected from the group comprising: Steinernema feltiae; Steinernema krussei and Steinernema carpocapsae.

In one embodiment, a formulation according to the invention is selected from the group consisting of:
(a) Heterorhabditis downesi, Heterorhabditis bacteriophora and Steinernema feltiae;
(b) Heterorhabditis downesi, Heterorhabditis bacteriophora and Steinernema carpocapsae;
(c) Heterorhabditis megidis, Heterorhabditis bacteriophora and Steinernema krussei;
(d) Heterorhabditis megidis, Heterorhabditis downsei and [symbol]
(e) Heterorhabditis downesi, Heterorhabditis megadis and Steinernema carpocapsae;
(f) Steinernema feltiae, Steinernema carpocapsae and Heterorhabditis downesi;
(g) Steinernema feltiae, Steinernema carpocapsae and Heterorhabditis besteriophora;
(h) Steinernema scapterisci, Steinernema carpocapsae and Heterorhabditis bacteriophora;
(i) Steinernema scapterisci, Steinernema carpocapsae and Heterorhabditis downesi;
(j) Steinernema feltiae, Steinernema carpocapsae and Heterorhabditis megidis; and
(k) Steinernema scapterisci, Steinernema carpocapsae and Heterorhabditis megidis;

In one embodiment of the invention, a formulation consisting essentially of Steinernema feltiae strain NY001, Steinernema carpocapsae strain Weiser, and Heterorhabditis bacteriophora strain Oswego, is disclaimed.

Suitably, the formulation additionally comprises a suitable media for the nematodes. Typically, the media is selected from the group comprising vermiculite, fine clay, water, and other types of suitable media. Generally, the media will comprise between 10 and 15% of the formulation.

Suitably, the cohort of nematodes will comprise at least 20%, 25%, 30%, or 33%, of the minority species. Thus, for example, in a cohort of three species including a single minority cohort will consist of the minority species. Ideally, the different species in the cohort will be present in approximately equal numbers (i.e. +/−5%).

The invention also relates to a method for the biological control of a predetermined target insect pest, which method employs a biological control formulation according to the invention in which the minority species in the formulation is parasitic to the predetermined target pest, the method comprising the step of applying the biological control formulation to the soil or other media in which the agricultural product is growing. Thus, in the case of a formulation consisting of Steinernema feltiae, Steinernema carpocapsae, and Heterorhabditis downesi, where the latter is the minority species, this formulation would be especially suitable for treating Root weevils, Wood borers, and/or Scarabs, as the minority species is especially parasitic to these insect pests. As indicated above, and in the data below, this formulation would have a far greater killing efficiency against the predetermined target pests than if the minority species were used on it's own. However, it should be noted that while the formulation would be especially suitable for treating the target insect pest, because the formulation includes two other species of nematode, it will also be effective in killing insect pests for which the latter two species of nematode are specialised killers. Thus, in the above example, the formulation would also provide effective action against a number of different insect pests, including Sciarid larvae, fungus gnats, armyworm, cutworm etc.

Thus, in one embodiment, the target insect is one or more of Sciarid larvae and Fungus gnats, and wherein the cohort of infective juvenile nematodes consists of Steinernema felitae and two species of Heterorhabditidae.

In another embodiment, the target insect is one or more of the Tawny mole cricket and the Southern mole cricket, and wherein the cohort of infective juvenile nematodes consists of Steinernema scapterisci and two species of Heterorhabditidae.

In another embodiment, the target insect is one or more of Root weevils, the Tawny mole cricket, and the Southern mole cricket, and wherein the cohort of infective juvenile nematodes consists of Steinernema riobravis, and two species of Heterorhabditidae.

In another embodiment, the target insect is a Root weevil, and wherein the cohort of infective juvenile nematodes consists of Heterorhabditis megidis, and two species of Steinernematidae.

In another embodiment, the target insect is one or more of Armyworms, Cutworms, Webworms, Root weevils, Wood borers, Artichokes, and Plume moths, and wherein the cohort of infective juvenile nematodes consists of Steinernema carpocapsae and two species of Heterorhabditidae.

In another embodiment, the target insect is one or more of Root weevils, Wood borers, and Scarabs, and wherein the cohort of infective juvenile nematodes consists of Heterorhabditis

[symbol]

The invention also relates to a method for effective the biological control of a target insect pest of an agricultural or horticultural product, which target pest predominantly inhabits an environment between 0 cm and 25 cm below surface level, which method employs a biological control formulation according to the invention, the method comprising the step of applying the biological control formulation to the soil or other media in which the agricultural or horticultural product is growing. A target pest that predominantly inhabits an environment between 0 cm and 25 cm below surface level is one in which at least 50%, ideally at least 75%, of the target pest population resides in this range of depths. Thus, for example, insect pests of mushrooms, strawberries, and most domestic and horticultural plants, would reside within this range of depths.

The invention also relates to a use of a formulation according to the invention to treat one or more of Sciarid larvae and Fungus gnats, wherein the cohort of infective juvenile nematodes consists of Steinernema felitae and two species of Heterorhabditidae.

In all of the above examples, the formulation would have a formidable killing effect against the target insect pest, and an effective killing effect against many other insect pests, for which the two species from the majority genus would be effective against. [symbol] to the invention to treat one or more of the Tawny mole cricket and the Southern mole cricket, and wherein the cohort of infective juvenile nematodes consists of Steinernema scapterisci and two species of Heterorhabditidae.

The invention also relates to a use of a formulation according to the invention to treat one or more of Root weevils, the Tawny mole cricket, and the Southern mole cricket, and wherein the cohort of infective juvenile nematodes consists of Steinernema riobravis, and two species of Heterorhabditidae.

The invention also relates to a use of a formulation according to the invention to treat a Root weevil, and wherein the cohort of infective juvenile nematodes consists of Heterorhabditis megidis, and two species of Steinernematidae.

The invention also relates to a use of a formulation according to the invention to treat one or more of Armyworms, Cutworms, Webworms, Root weevils, Wood borers, Artichokes, and Plume moths, and wherein the cohort of infective juvenile nematodes consists of Steinernema carpocapsae and two species of Heterorhabditidae.

The invention also relates to a use of a formulation according to the invention to treat one or more of Root weevils, Wood borers, and Scarabs, and wherein the cohort of infective juvenile nematodes consists of Heterorhabditis bacteriophora and two species of Steinernamitidae. [symbol] is provided a formulation for use in the biological control of insect pests comprising a cohort of infective juvenile insect-parasitic nematodes from at least three species, wherein at least two of the species are of a first genus, and at least one of the species are from a second genus, wherein the number of species from the first genus is greater than the number of species from the second genus, and wherein the formulation comprises at least one ambusher foraging species of nematode and at least one foraging species of nematode, or at least one nematode species that is capable of both cruiser and ambusher foraging strategies.

The invention also relates to a packaged product comprising a formulation according to the invention, the packaged product comprising information indicating the identity of the predetermined target insect pest (or pests), wherein the minority species in the formulation is a specialised parasite to the predetermined target insect pest (or pests). Typically, the packaged product further comprises instructions for a user to apply the formulation to a desired ecosystem. Suitably, the formulation in the packaged product additionally comprises a suitable media for the nematodes. Typically, the media is selected from the group comprising vermiculite, fine clay, sponge, water, and other types of suitable media. Generally, the media will comprise between 10 and 15% of the formulation.

The invention also relates to a method of formulating a cohort of infective juvenile insect-parasitic nematodes for use in the biological control of a predetermined target insect pest [symbol] the invention, wherein the minority species in the formulation is a specialised parasite to the predetermined target insect pest (or pests).

Table I provides a list of nematode species along with a list of the target pests for which the nematode species is a specialised parasite.

### DETAILED DESCRIPTION OF THE INVENTION

The Steinernema (Steinernematidae) and Heterorhabditis (Heterorhabditidae) genera contain several species, each specialised for attacking a specific type or group of insect pests (Table I). The formulations of the invention, by virtue of comprising at least three different species of nematode, thereby provide a broad spectrum effect against a number of types and classes of insect pests.

TABLE I
Current use of beneficial nematodes as biological control agents

| | Foraging | | |
|---|---|---|---|
| Nematode Species | strategy | Insect pest | Commodity |
| Steinernema feltiae | both ambusher | Sciarid larvae, | Mushrooms, |
| | and cruiser | Fungus gnats. | Ornamentals |
| Steinernema scapterisci | ambush forager | Tawny mole cricket, | Turf grass |
| | | Southern mole | |
| Steinernema riobravis | both ambusher | Root weevils, Tawny mole | Citrus, |
| | and cruiser | cricket, Southern mole | Turf grass |
| | | cricket. | |
| Steinernema carpocapsae | ambush forager | Armyworm, Cutworm, | Ornamentals, |
| | | Webworm, Root weevils | Cranberries |
| | | Wood borers, Artichokes | |
| | | plume moth. | |
| Steinernema glaseri | cruiser forager | coleopterous larvae, | Citrus, |
| | | particularly scarabs | Turf grass |
| Steinernema kraussei | cruiser forager | Vine weevil. | Gardens, |
| | | | Ornamenals |
| Heterorhabditis megidis | cruiser forager | Root weevils. | Ornamentals |
| Heterorhabditis bacteriophora | cruiser forager | Root weevils, Wood | Ornamentals, |
| | | borers, Scarabs. | Berries, |
| | | | Turf grass |

[symbol] invention include:

Steinernema Species

Steinernema oregonense, Steinernema anomaly, Steinernema intermedia, Steinernema rarum, Steinernema kushidai, Steinernema abbasi, Steinernema bicomutum, Steinernema siamkayai, Steinernema arenarium, Steinernema cubanum, Steinernema glaseri, Steinernema karii, Steinernema puertoricense.

Heterorhabditis Species

Heterorhabditis Hawaiians, Heterorhabditis indicus, Heterorhabditis zealandica, Heterorhabditis downesi, Heterorhabditis marelatus, Heterorhabditis baujardi, Heterorhabditis floridness, Heterorhabditis Mexicana.

Methods for Producing Infective Juvenile Insect-Parasite Nematodes

For small-scale field-testing, in vivo production (as described by Woodring and Kaya (1988)-“Steinernematids and Heterorhabditids Nematodes: Handbook of Biology and Techniques”. Southern Cooperative Bulletin 331, Arkanass Agricultural Experimental Station, Fayettville, Ark., 30 pp.), is used to produce a large number of non-feeding infective juveniles.

Infecting: [symbol] or Galleria larvae) with infective juveniles of the beneficial nematodes (steinernematids or heterorhabditids). The infective juveniles are first left to warm to room temperature (20-25° C.) in an aqueous suspension. Then a selected number of these nematode juveniles are transferred to specially prepare plastic containers in which a sheet of blotting paper is placed inside. Next the insect larvae are added to give a ratio of some twenty nematodes for every insect larva. The lid is replaced and the container is incubated at 25° C. for some six to eight days.

Harvesting the New Generation of Juvenile Nematodes:

The infected insect larvae are transferred to a water trap and over a period of 10-12 days, following infection, the new juveniles migrate from the host larvae into the water. Soon after they appear they can be easily harvested. The harvested nematodes are surface sterilised and washed several times before they are mixed together. The sterilant used comprises 0.1% Milton solution and washing is carried out using distilled water.

After harvesting, the nematodes are packed in vermiculite, and packaged in plastic containers which are sealed with a permeable film for storage and transport. However, other media and packaging forms may be employed, and suitable examples of such media and packaging will be well known to those skilled in the art.

This formulation of the invention may be used to control insect pest species found in domestic greenhouses and gardens, and in commercial greenhouses, farms and forrests. The formulation may be especially applicable for use with nursery stocks, turf grass, strawberries, cranberries, indoor or outdoor pot plants or grow bags suffering from soil insect pests attacks, and garden insect pests.

Specific examples of formulation according to the invention are provided below:

A—Formulations: 2 cruiser forager+1 ambush forager

A1: Heterorhabditis megidis, Heterorhabditis bacteriophora and Steinernema feltiae

This formulation would control insect pests selected from the group comprising Dipterous insects, including mushroom flies, fungus gnats, and tipulids, Lepidopterous which includes Armyworm, Cutworm, webworms, Caterpillar borers, Coleopterous insect larvae, Root weevils, Citrus weevils, Black vine weevils, chafers, Scarabs, Hymenoptera, and queen ants/termites.

A2: Heterorhabditis megidis, Heterorhabditis bacteriophora and Steinernema carpocapsae

This formulation would be capable of controlling Dipterous insects, including fungus gnats, and tipulids, Lepidopterous which may includes Armyworm, Cutworm, webworms, Caterpillar weevils, Black vine weevils, chafers, Scarabs, Hymenoptera, and queen ants/termites

B-Formulations: 2 ambush forager+1 cruiser forager

B1: Steinernema feltiae, Steinernema carpocapsae+Heterorhabditis megidis

This formulation is capable of controlling Dipterous insects, including mushroom flies, fungus gnats, and tipulids, Lepidopterous which may includes Armyworm, Cutworm, webworms, Caterpillar borers, Coleopterous insect larvae, Root weevils, Citrus weevils, Black vine weevils, chafers, Scarabs, and Hymenoptera, queen ants/termites.

B2: Steinernema scapterisci, Steinernema carpocapsae+Heterorhabditis bacteriophora

This formulation is capable of controlling Dipterous insects, including fungus gnats, and tipulids, Lepidopterous which may includes Armyworm, Cutworm, webworms, Caterpillar borers, Coleopterous insect larvae, Root weevils, Citrus weevils, Black vine weevils, chafers, Scarabs, Hymenoptera, queen ants/termites, and Orthoptera, including the Tawny mole cricket and the Southern mole cricket.

Application for Formulations A: (Based on small package 30 millions of infective juvenile nematodes)

As nematodes need moisture in the soil for movement (if the soil is too dry or compact, they may not able to search out immediately after applying the formulation of the invention is advised, as it keeps the soil moist and helps the juvenile nematodes to move deeper into the soil. Care should be taken not to soak the area because nematodes in too much water cannot infect. There is no need for masks or specialized safety equipment, as the formulations of the invention are safe for plants and animals (birds, pets, children).

Mixed with Compost.
A Before sowing or planting, or
B While the crop is growing.

The formulations of the invention may be applied to the ground before sowing or planting (in which case they are known as base dressing) or while the crop is growing, as top dressing. Dosage: 1 box of the formulation, mixed well with 160-200 litres of loose moist compost, which may be used in base dressing or in top dressing. Similar application may be applied for making out or in-door pots.

Insert in Pots or Grow Bags.

Insert 1 spoon (one gram) of the formulated invention product in each pot; treating up to 200 pots (1 litre pot) or 3 spoons in each grow bag treating up to 60-70 grow bags.

Insert in the Soil.

Scoop the soil to make a small holes 5-10 cm deep (using a small trowel) and 1 meter apart a around the garden. Insert 2 spoons of the formulation in each hole and gently firm the soil with the hand.

Chemical Fertilizers should be avoided roughly 2 weeks prior to and after nematode application, because they may be adversely affected by high nitrogen content.

Storage and Handling.

In general, formulations of the invention should be stored in a cool refrigerator 2-5° C., and should not be allowed to freeze.
1 Keep in a dry place out of direct sunlight and not to be exposed to extreme temperatures.
2 Mix all the ingredients (contents) gently before use and keep away from direct sunlight.
3 Use Before Expiry Date.

Application for Formulations B: (Based on medium package 45 millions of infective juvenile nematodes).

Open a pack of the formulation of the invention and empty the entire contents (nematodes and fine soil) into a bucket or watering can, containing 4-5 litres of tap water. Mix well and leave it for 5-10 minutes to soak, mix well again and pour the entire content through a fine sieve into a spray tank and adjust to the required amount of water, stir well and spray onto the target area immediately.

Formulations of the invention may be packaged in various quantities, including:

A—Small size containing 30 millions of infective juvenile treat 160-200 small pots or 60-70 square metres.

B—Standard size containing 45 millions of infective juvenile nematodes, may treat 100 square metres.

C—Large size containing 90 millions of infective juvenile nematodes fine soil (as inert caring, may treat 200 square metres.

Two family, Steinernematidae and Heterorhabditidae, from order Rhabditida entomopathogenic nematodes have been used as most effective bio-control against wide range of soil inhabiting insect pest.

The most important key, amongst others, to successful pest control with these beneficial nematodes is:
matching the correct nematode species with the pest species;

So one of the major problems which arise with the use of the existence of the biological control product, Steinernematids species or Heterorhabditids species, sold in the market, contain only one species of the above beneficial nematodes which may able to control only a single or one group of target insect species, make it uneconomically viable, if there are a multi pest species in the field, comparing with the use of chemical pesticide which may control a wide range of insect species in one application.

Many researcher have studied the possibility of using more than one species of beneficial nematodes, to control insect pests. In general the majority of them concentrate on the use of different foraging strategies ie cruisers (widely foraging) and ambusher (sit and wait).

There are several product sold in the marked such as Nematak (ambusher)-NemaSeek (cruiser) Combo (one pack for Nematak and one for NemaSeek) and others. All products claim they are more effective for the control of a wider range of insect pest over a single species application.

Experimental

Several trials were set-up to compare between the foraging strategies and majority and minority species (two species from Heterorhabditis genus and one species from Steinernema genus or two species from Steinernema genus and one species from Heterorhabditis genus) to control several insect larvae and pupae in general multipurpose compost.

All the trial were conducted in FITZGERALD NURSERIES Ltd. OLDTOWN, STONEYFORD, Co. KILKENNY, REPUBLIC of IRELAND.

I—Using 2 species from the genus Steinernema and 1 species from the genus Heterorhabditis.

Material and Methods

Source of beneficial nematodes used for all the trials below. Steinernema feltiae and Steinernema kraussei were obtained from Nemasys dealer in Ireland, Heterorhabditis downesi were supplied by Dr. C. Griffin, Maynooth College, Co. Kildare, Ireland and Steinernema carpocapsae, Heterorhabditis bacteriophora and Heterorhabditis megidis were obtained from Koppert agent in Ireland. [symbol] with 10 litre multi-purpose compost. Each pot inserted with three plastic mesh pouches, each pouch containing 9 insect larvae (2 root vine weevils, 2 galleria larvae, 5 meal worm larvae), one on the top (1-2 centimetres below the surface), one in the middle (12 centimetres below the surface) and one in the bottom of the pot (24 centimetres below the surface). A 10 ml aliquot nematode suspension, 60000 IJs, were spread evenly on the surface of each pot in the appropriate treatment, the application rate is the same for the single species (60000), two species (30000 for each species=600000) and in 3 species novel application (20000 for each species=60000). The above pots were divided in three group of 10 pots and used in the following trials treatments:

Trial 1

A—Using 1 species from the genus Heterorhabditis and 2 species from the genus Steinernema resemble three foraging strategies.
1—Untreated Control
2—Heterorhabditis downesi alone
3—Steinernema feltiae, Steinernema carpocapsae and Heterorhabditis downesi.

These three species will resemble three foraging strategies, cruiser (Heterorhabditis downesi), intermediate (Steinernema feltiae) and ambusher (Steinernema carpocapsae).
1—No nematodes were added to untreated control.
2—Heterorhabditis downesi 10×60,000=600,000
3—Steinernema feltiae 20×20,000=200,000
3—Steinernema carpocapsae 10×20,000=200,000
3—Heterorhabditis downesi 10×20,000=200,000

Ten millilitres of tap water were added to each pot of the untreated control.

A 10 ml aliquot nematodes, Heterorhabditis downesi, suspension (60000 IJs) were added evenly on top of each pot in the second treatment and similarly 60000 IJs of novel mix species (20000 Steinernema feltiae, 20000 Steinernema carpocapsae and 20000 Heterorhabditis downesi).

B—Using 1 species from the genus Steinernema and 2 species from the genus Heterorhabditis resemble two foraging strategies.

The trial were repeated with other combination one ambusher, Steinernema carpocapsae, and two cruisers, Heterorhabditis downesi and Heterorhabditis bacteriophora
1—Untreated Control
2—Steinernema carpocapsae alone
3—Heterorhabditis downesi, Heterorhabditis bacteriophora and Steinernema carpocapsae.
1—No nematodes were added for untreated control.
3—Steinernema carpocapsae 10×20,000=200,000
3—Heterorhabditis bacteriophora 10×20,000=200,000

Similarly, ten millilitres of tap water were added to each pot of the untreated control.

A 10 ml aliquot nematodes, Steinernema carpocapsae, suspension (60000 IJs) were added evenly on top of each pot in the second treatment and similarly 60000 IJs of novel mix species (20000 Heterorhabditis downesi, 20000 Heterorhabditis bacteriophora and 20000 Steinernema carpocapsae).

All the pots (for both trials) were incubated in a polythene tunnel in FITZGERALD NURSERIES for 7 days at temperatures between 6 to 18° C. (air temperatures). On day 7 all the plastic mesh pouches were removed separately for each pot as top middle and bottom and all the dead insect were examined visually if the changing colour mach the specific beneficial nematode which may suggested to which nematodes species infected the host (insect larvae or pupae), or the dead insect were dissected under the stereoscopic microscope for the present the beneficial nematodes.

Results and Discussion.

As can be seen from Table 1 when applied Heterorhabditis downesi alone manage to infect insect species at all levels (Top, middle and bottom) more on the top and at lesser extend at the lower levels. However in the case of vine weevils it known to target coleopteran larvae especially pine weevils (Dr. C. Griffin personal communication). The use of mealworm larvae is to make it more challenging for H. downesi as they have more tougher cuticle. Galleria larvae were used in all combinations to indicate which level the nematodes can travel deep in the pot, as these larvae have no immunity, make them an easy target by all the nematodes used in these trials. However, when H. downesi were used in the novel multi-species application, 2 Steinernematids species and one Heterorhabditids species, one ambusher, one intermediate (ambusher and cruiser) and one cruiser [Steinernema carpocapsae (ambusher), Steinernema feltiae (cruiser and ambusher) and Heterorhabditis downesi (cruiser)], this combination manage to increase the infection by nearly between 50-100% (see Table 2) and followed the same patron as in Table 1, higher on the top and lower on the middle and the bottom but still higher than the single species application by at lease 50-100%.

TABLE 1
Percentage of insect larvae attacked
by Heterorhabditis downesi alone.

| | Vine weevils | Meal worm | Galleria |
|---|---|---|---|
| Location | larvae | larvae | larvae |
| Top | 10/20 = 50% | 19/40 = 47.5% | 15/20 = 75% |
| Middle | 3/20 = 15% | 22/40 = 55% | 10/20 = 50% |
| Bottom | 5/20 = 25% | 11/40 = 27.5% | 9/20 = 45% |
| Total | 18/60 = 30% | 52/120 = 43.35% | 34/60 = 56.75% |

General Total 104/240 = 43.3%

TABLE 2
Percentage of insect larvae attacked by a novel multi-species
application (Steinernema carpocapsae, Steinernema feltiae
and Heterorhabditis downesi).

| | Vine weevils | Mealworm | Galleria |
|---|---|---|---|
| Location | larvae | larvae | larvae |
| Top | 16/20 = 80% | 39/40 = 97.55% | 20/20 = 100% |
| Middle | 17/20 = 85% | 35/40 = 87.55% | 20/20 = 100% |
| Bottom | 12/20 = 60% | 29/40 = 75.55% | 19/20 = 95% |
| Total | 45/60 = 75% | 103/120 = 85.8% | 59/60 = 98.35% |

General Total 207/240 = 86.3%

Steinernema carpocapsae usually used to control coleopteran (Root weevils, Billbugs) larvae and pupae and also recommended to control lepidopteron larvae (Artichoke plume moth, Armyworm, Cutworm, Webworm). In the second trial 2 vine weevils, 5 mealworm pupae, 5 mealworm larvae and 2 galleria larvae were used in this trial.

Table 3 show that Steinernema carpocapsae followed the same patron as in H. downesi when applied alone Table 1. The highest attack on the top and lowest at the bottom. However when used in multi species, 2 Heterorhabditids species and one Steinernematids species, two cruiser and one ambusher (H. downesi (cruiser), H. bacteriophora (cruiser), and S. carpocapsae (ambusher). Table 4 also show a higher infected larvae and pupae (more than 30-100%) although the total attack is less the in H. downesi, multi-species application Table 4, is still nearly 50% improvement than the single S.

TABLE 3
Percentage of insect larvae attacked by S. carpocapsae alone.

| | Vine weevils | Meal worm | Meal worm | Galleria |
|---|---|---|---|---|
| Location | larvae | pupae | larvae | larvae |
| Top | 14/20 = 70% | 33/50 = 66% | 31/50 = 62% | 13/20 = 65% |
| Middle | 9/19 = 47.4% | 20/50 = 40% | 13/50 = 26% | 8/20 = 40% |
| Bottom | 4/19 = 21.1% | 22/50 = 44% | 17/50 = 34% | 11/20 = 55% |
| Total | 27/58 = 46.5% | 75/150 = 50% | 61/150 = 41% | 32/60 = 53% |

General Total 195/365 = 53.4%

TABLE 4
Percentage of insect larvae attacked by a novel multi-species application
(H. downesi, H. bacteriophora and S. carpocapsae)

| | Vine weevils | Meal worm | Meal worm | Galleria |
|---|---|---|---|---|
| Location | larvae | pupae | larvae | larvae |
| Top | 13/20 = 65% | 42/50 = 84% | 39/50 = 78% | 17/20 = 85% |
| Middle | 15/20 = 75% | 35/50 = 70% | 34/50 = 68% | 17/20 = 85% |
| Bottom | 14/20 = 70% | 33/50 = 66% | 28/50 = 56% | 16/20 = 80% |
| Total | 42/60 = 70% | 110/150 = 73.3% | 101/150 = 67.3% | 50/60 = 83.3% |

General Total 303/420 = 72%

Material and Methods

The second trial were conducted also in FITZGERALD NURSERIES with different combinations and similarly incubated in polythene tunnel at for 6 days at temperatures between 7 to 15° C. On day 6 all the plastic mesh pouches were removed separately for each pot as top middle and bottom and all the dead insect were examined visually if the changing colour mach the specific beneficial nematode which may suggested to which nematodes species infected the host (insect larvae or pupae), or the dead insect were dissected under the stereoscopic microscope for the present the beneficial nematodes.

C—Using 1 species from the genus Steinernema and 2 species from the genus Heterorhabditis resemble only one foraging strategies.

Steinernema kraussei usually used to control coleopteran, pine weevils, vine weevils.

In this trial 5 maggot larvae, 5 mealworm pupae, 5 mealworm larvae and 3 galleria larvae were used in this trial.

Results and Discussion.

Table 5 show that S. kraussei followed the same patron as in H. downesi and in S. carpocapsae, when it applied alone Table 1 and Table 3. The highest attack on the top and lowest at the minority), 2 Heterorhabditids species and one Steinernematids species, all represent cruiser foragers. (H. downesi (cruiser), H. megidis (cruiser) and S. kraussei (cruiser). Table 5 also show a higher infected larvae and pupae (more than 30-100%) although the total attack is less the in H. downesi, multi-species application, Table 6, is still nearly 50% improvement than the single species (see Tables 5 & 6).

TABLE 5
Percentage of insect larvae attacked by S. kraussei alone

| | Maggot | Mealworm | Mealworms | Galleria |
|---|---|---|---|---|
| Location | larvae (5) | pupae (5) | larvae (5) | larvae (3) |
| Top | 18/44 = 40.9% | 36/50 = 72% | 48/50 = 96% | 25/30 = 83.3% |
| Middle | 7/43 = 16.3% | 34/50 = 68% | 37/50 = 74% | 27/30 = 90% |
| Bottom | 3/35 = 8.8% | 21/50 = 42% | 0/50 = 0% | 8/30 = 23.3% |
| Total | 28/122 = 22.9 | 91/150 = 60.7% | 85/150 = 56.7% | 60/90 = 66.7% |

General Total 264/512 = 51.6%

Percentage of insect larvae attacked by a novel multi-species application (H. downesi, H. megidis and S. kraussei)

| | Maggot | Mealworm | Mealworms | Galleria |
|---|---|---|---|---|
| Location | larvae (5) | pupae (5) | larvae (5) | larvae (3) |
| Top | 22/41 = 53.6% | 40/50 = 80% | 50/50 = 100% | 30/30 = 100% |
| Middle | 10/42 = 23.8% | 45/50 = 90% | 49/50 = 98% | 30/30 = 100% |
| Bottom | 13/44 = 29.5% | 40/50 = 80% | 13/50 = 26% | 30/30 = 100% |
| Total | 45/127 = 35.4% | 125/150 = 83.3% | 112/150 = 74.7% | 90/90 = 100% |

General Total 372/517 = 72%

D—Using 1 species from the genus Steinernema and 2 species from the genus Heterorhabditis resemble “two foraging strategies”.

Steinernema feltiae usually used to control dipterans larvae, glasshouse sciarids and destroys sciarid larvae in mushroom compost.

In this trial 5 maggot larvae, 5 mealworm pupae, 5 mealworm larvae and 3 galleria larvae were used in this trial.

Results and Discussion.

Table 7 show that S. feltiae followed the same patron as in H. downesi Table 1, S. carpocapsae Table 3 and S. kraussei Table 5, when they applied alone. The highest attack on the top and lowest at the bottom. However when used in multi species, 2 Heterorhabditids species and one Steinernematids ambusher) (H. downesi (cruiser), H. megidis (cruiser) and S. feltiae [intermediate (cruiser and ambusher)]. Table 8 also show a higher infected larvae and pupae (more than 30-100%) the total attack in S. feltiae, multi-species application Table 8, is still more than 70% improvement than the single species application (see Tables 7).

TABLE 7
Percentage of insect larvae attacked by Steinernema feltiae alone.

| | Maggot | Mealworm | Mealworms | Galleria |
|---|---|---|---|---|
| Location | larvae (5) | pupae (5) | larvae (5) | larvae (3) |
| Top | 10/36 = 27.8% | 40/50 = 80% | 45/50 = 90% | 22/30 = 73.3% |
| Middle | 6/32 = 35.3% | 25/50 = 50% | 7/50 = 14% | 10/30 = 33.3% |
| Bottom | 4/37 = 10.8% | 20/50 = 40% | 7/50 = 16% | 3/30 = 10% |
| Total | 20/105 = 19% | 85/150 = 56.7% | 59/150 = 39.3% | 35/90 = 38.9% |

General Total 199/495 = 40.2%

Percentage of insect larvae attacked by Steinernema feltiae, multi-species application (H. downesi, H. megidis and S. feltiae).

| | Maggot | Mealworm | Mealworms | Galleria |
|---|---|---|---|---|
| Location | larvae (5) | pupae (5) | larvae (5) | larvae (3) |
| Top | 12/39 = 30.8% | 39/50 = 78% | 50/50 = 100% | 25/30 = 83.3% |
| Middle | 19/45 = 42.2% | 49/50 = 98% | 40/50 = 80% | 27/30 = 90% |
| Bottom | 11/33 = 33.3% | 40/50 = 80% | 20/50 = 40% | 25/30 = 83.3% |
| Total | 42/117 = 35.9% | 128/150 = 85.3% | 110/150 = 73.3% | 77/90 = 85.5% |

General Total 357/507 = 70.4%

In general found the attack by each species, of beneficial nematodes tried above, mainly on the top and lesser for the deeper attack. All the nematodes used appear to have the capability to use different foraging strategies specially in the case of S. carpocapsaeas as an ambusher found to be able to roam deeper in the compost and attack host at all locations (top, middle and bottom) Tables 1-8. However the majority and minority combination, increase the attack by 30-100% at all levels and by all the nematodes species used here. The foraging behaviours made no different in the majority and minority combinations, in all the species combinations used, ambusher, intermediate (cruiser and ambusher) and (cruiser) Table 2, two cruiser and one ambusher Table 4, all represent cruiser foragers Table 6 and two cruiser and one intermediate (cruiser and ambusher) Table 8. All the species are present [symbol] a higher on top and middle and lesser on the bottom but there are a higher increase in the attack more than double comparing with the single species (see Tables 1-8).

Other trials were conducted to assess the benefit of the use of two species from the same genus with different or same foraging strategies.

As can been seen from Table 9 the beneficial nematodes (S. Krause (cruiser)) and S. feltiae (intermediate) manage to attack the host at all levels and followed the same patron as in single species and in novel combination. However the level of the attack as low as in the single species (see Tables 1, 3, 5 and 7).

TABLE 9
Percentage of insect larvae attacked by Steinernema kraussei
and Steinernema feltiae

| | Maggot | Mealworm | Mealworms | Galleria |
|---|---|---|---|---|
| Location | larvae (5) | pupae (5) | larvae (5) | larvae (3) |
| Top | 14/50 = 28% | 38/50 = 76% | 39/50 = 78% | 23/30 = 76.7% |
| Middle | 10/46 = 21.7% | 36/50 = 72% | 44/50 = 88% | 22/30 = 73.3% |
| Bottom | 5/48 = 10.4% | 27/50 = 54% | 3/50 = 6% | 20/30 = 66.7% |
| Total | 29/144 = 20% | 101/150 = 67.3 | 86/150 = 57% | 65/90 = 72.2% |

General Total 281/534 = 52.6%

[symbol] (cruiser)) and S. carpocapsae (ambusher) manage to attack the host at all levels and similarly followed the same patron as in single species and in novel combination. However the level of the attack as low as in the single species (see Tables 1, 3, 5 and 7).

TABLE 10
Number of insect larvae attacked by S. kraussei and S. carpocapsae.

| | Maggot | Mealworm | Mealworms | Galleria |
|---|---|---|---|---|
| Location | larvae (5) | pupae (5) | larvae (5) | larvae (3) |
| Top | 8/38 = 21% | 32/50 = 64% | 46/50 = 92% | 20/30 = 66.7% |
| Middle | 6/49 = 12.2% | 24/50 = 48% | 29/50 = 58% | 21/30 = 70% |
| Bottom | 2/45 = 4.4% | 27/50 = 54% | 2/50 = 4% | 18/30 = 60% |
| Total | 16/132 = 12.1% | 83/150 = 55.5% | 77/150 = 51.3% | 59/90 = 65.5% |

General Total 235/522 = 45%

Table 11 also shows that the beneficial nematodes, H. downesi (cruiser) and H. megidis (cruiser) manage to attack the host at all levels and similarly followed the same patron as in single species and in novel combination. However the level of the attack as low as in the single species (see Tables 1, 3, 5 and 7).

Number of insect larvae attacked by H. downesi and H. megidis.

| | Maggot | Mealworm | Mealworms | Galleria |
|---|---|---|---|---|
| Location | larvae (5) | pupae (5) | larvae (5) | larvae (3) |
| Top | 12/43 = 27.9% | 37/50 = 74% | 13/50 = 26% | 23/30 = 76.7% |
| Middle | 11/50 = 22% | 37/50 = 74% | 17/50 = 34% | 22/30 = 44% |
| Bottom | 5/50 = 10% | 22/50 = 44% | 4/50 = 8% | 12/30 = 40% |
| Total | 28/143 = 19.6% | 96/150 = 64% | 34/150 = 22.7% | 57/90 = 63.3% |

General Total 215/533 = 40.3%

The result from Tables 9, 10 and 11 suggest there is no benefit to using combinations consisting of species form the same genus. Kaya and Koppenhofer (1996) stated that this competition can reduce nematode fitness and can cause local extinction of a nematodes species.

To confirm, the advantages of this invention over the prior art includes the following points:
(i) The minority species is more effective in a multi species formulation of the invention than by itself. This was found to be the case at all depths.
(ii) The formulation of this invention is independent of foraging strategy utilised by the nematode as the killing effect is strong when the same foraging strategy (all cruiser or all ambusher) or different foraging strategies are tested.
(iii) The timing of the introduction of the beneficial nematodes and host species by Neumann et al. (2006) is different from the formulation of this invention.

The invention is not limited to the embodiments hereinbefore described which may be varied in both construction and detail without departing from the spirit of the invention.

### REFERENCES CITED

Kaya, H. K., and A. M. Koppenhofer (1996). Coexistence of entomopathogenic nematode species (Steinernematidae and Heterorhabditidae) with different foraging behavior. Fundam. Appl. Nemattol. 19: 175-183.

Neumannn, C. and E. J. Shields. 2006. Interspecific Interractions Among Three Entomopathogenic Nematodes, Steinernema carpocapsae Weiser, Steinernema feltiae Filipjev, and Heterorhabditis bacteriophora Poinar, with Different Foraging Strategies for Hostsin Multipiece sand Columns. Environ. Entomol. 35 (6): 1576-1583.

1-40. (canceled)

41. A formulation for use in the biological control of insect pests comprising a cohort of infective juvenile insect-parasitic nematodes and a media for the nematodes, the cohort comprising a mixture of at least three species in which two of the species are of a first genus and one of the species is from a second genus, and wherein the number of species from the first genus is greater than the number of species from the second genus.

42. The formulation as claimed in claim 41 in which the cohort comprises (a) at least one cruiser foraging species and at least one ambusher foraging species, or (b) a nematode that is capable of both cruiser and ambusher foraging strategies.

43. The formulation as claimed in claim 41 in which the cohort of infective juvenile nematodes consists of two or more species from the first genus and one species from the second genus.

44. The formulation as claimed in claim 43 in which the cohort consists of 3, 4, 5, 6, 7, 9, or 10 species from the first genus, and one species from the second genus.

45. The formulation as claimed claim 41 in which the cohort of infective juvenile nematodes consists of two species from the first genus and one species from the second genus.

46. The formulation as claimed in claim 41 in which the first and second genus are selected from the group consisting of: Steinernema; and Heterorhapditis.

47. The formulation as claimed in claim 46 in which the first genus is Steinernema and the second genus is Heterorhapditis.

48. The formulation as claimed in claim 46 in which the first genus is Heterorhapditis and the second genus is Steinernema.

49. The formulation as claimed in claim 41 in which the species of Heterorhapditis is/are selected from the group comprising: Heterorhabditis megidis; Heterorhabditis downesi and Heterorhabditis bacteriophora.

50. The formulation as claimed in claim 41 in which the species of Steinernema is/are selected from the group comprising: Steinernema feltiae; Stinernema scapterisci; Steinernema riobravis; Steinernema carpocapsae; and Steinernema krussei.

51. The formulation as claimed in claim 41 in which the cohort of infective juvenile insect-parasitic nematodes is selected from the group consisting of:
(a) Heterorhabditis downesi, Heterorhabditis bacteriophora and Steinernema feltiae;
(b) Heterorhabditis downesi, Heterorhabditis bacteriophora and Steinernema carpocapsae;
(c) Heterorhabditis megidis, Heterorhabditis bacteriophora and Steinernema krussei;
(d) Heterorhabditis megidis, Heterorhabditis downesi and Steinernema krussei;
(e) Heterorhabditis downesi, Heterorhabditis megadis and Steinernema carpocapsae;
(f) Steinernema feltiae, Steinernema carpocapsae and Heterorhabditis downesi;
(g) Steinernema feltiae, Steinernema carpocapsae and Heterorhabditis bacteriophora;
(h) Steinernema scapterisci, Steinernema carpocapsae and Heterorhabditis bacteriophora;
(i) Steinernema scapterisci, Steinernema carpocapsae and Heterorhabditis downesi;
(j) Steinernema feltiae, Steinernema carpocapsae and Heterorhabditis megidis; and
(k) Steinernema scapterisci, Steinernema carpocapsae and Heterorhabditis megidis.

52. The formulation as claimed in claim 51 in which the media is selected from the group comprising vermiculite, fine clay, water, and other types of suitable media.

53. A method for the biological control of a predetermined target insect pest, which method employs a biological control formulation according to claim 41, and in which the second genus in the formulation is a specialised parasite against a predetermined target insect pest, the method comprising the step of applying the biological control formulation to soil or other media in which an agricultural product is growing.

54. The method as claimed in claim 53 in which the target insect is one or more of Sciarid larvae and Fungus gnats, and wherein the cohort of infective juvenile nematodes consists of Steinernema felitae and two species of Heterorhabditis.

55. The method as claimed in claim 53 in which the target insect is one or more of the Tawny mole cricket and the Southern mole cricket, and wherein the cohort of infective juvenile nematodes consists of Steinernema scapterisci and two species of Heterorhabditis.

56. The method as claimed in claim 53 in which the target insect is one or more of Root weevils, the Tawny mole cricket, and the Southern mole cricket, and wherein the cohort of infective juvenile nematodes consists of Steinernema riobravis, and two species of Heterorhabditis.

57. The method as claimed in claim 53 in which the target insect is a Root weevil, and wherein the cohort of infective juvenile nematodes consists of Heterorhabditis megidis, and two species of Steinernema.

58. The method as claimed in claim 53 in which the target insect is one or more of Armyworms, Cutworms, Webworms, Root weevils, Wood borers, Artichokes, and Plume moths, and wherein the cohort of infective juvenile nematodes consists of Steinernema carpocapsae and two species of Heterorhabditis.

59. The method as claimed in claim 53 in which the target insect is one or more of Root weevils, Wood borers, and Scarabs, and wherein the cohort of infective juvenile nematodes consists of Heterorhabditis bacteriophora and two species of Steinernema.

60. A packaged product comprising a formulation according to claim 41.
